(19) United States
(12) Patent Application Publication
(10) Pub. No.: US 2025/0364147 A1
(43) Pub. Date: Nov. 27, 2025

(54) SYSTEMS AND METHODS FOR MULTIMODALITY FUSION OF MEDICAL DATA SOURCES

(71) Applicant: Wisconsin Alumni Research Foundation, Madison, WI (US)

(72) Inventors: Olivia Krebs, Madison, WI (US); Pallavi Tiwari, Madison, WI (US)

(21) Appl. No.: 18/674,796

(22) Filed: May 24, 2024

(51) Int. Cl. G16H 50/70; G16H 15/00; G16H 50/20

(52) U.S. Cl. CPC G16H 50/70; G16H 15/00; G16H 50/20

(57) ABSTRACT

Systems and methods are provided for creating report using the integrated prognostic signature indicating at least one of a diagnosis of the disease or a response of a subject to a treatment. The method may include receiving, by a system comprising a processor, a plurality of datasets from different modalities associated with a disease of a subject and determining, by the processor, a matrix of features for each of the plurality of data modalities. The method may also include integrating, by the processor, the matrix of features through a sequential, hierarchical structure to create an integrated prognostic signature for the subject, creating, by the processor, a report using the integrated prognostic signature indicating at least one of a diagnosis of the disease or a response of the subject to a treatment and displaying, by the system, the report.

### STATEMENT OF GOVERNMENT SUPPORT

N/A.

### BACKGROUND

Modern healthcare relies upon a variety of information and diagnostic sources to empower clinicians to make decisions. Cancer diagnosis, treatment, and prognosis is a complex task involving the acquisition of complementary data streams including imaging scans (e.g. CT or MRI images), molecular data (e.g., genome sequencing, gene-expression, epigenomics, also known as multi-omics), cellular-scale (e.g., histological images), as well as clinical data (such as age, gender, cognitive scores). Detailed, comprehensive assessment of the deluge of data is often infeasible for clinicians alone. The recent increase in the performance, costs, and access to sophisticated computational resources has presented an opportunity to supplement the analysis of information by the clinician with algorithmic or deep learning information synthesis.

Current deep learning approaches for prognostic modeling generally seek to retrospectively mirror the analysis performed by the clinician and improve the analysis by identifying diagnostic information that could be missed by the clinician. For example, a clinician may analyze medical images, such as MR or CT images, to identify tumors. Deep learning systems can retrospectively analyze those same images and highlight potential indicators of tumors that may be, for example, too small for the clinician to identify, or assist with defining tumor margins that may be difficult to discern. These systems find clinical utility because they can be readily trained from available datasets, such as images of patients confirmed to have cancer, that serve as a clear and understandable ground truth.

Some have extended this aided analysis to combine multiple modalities of biomedical data. Again, these efforts seek to retrospectively follow the clinician's analysis process because it is understood by the clinicians that will use the system and because doing so provides clear and available datasets to serve as ground truth. For example, like a clinician that may consider an image of a breast showing a tumor and genetic information about the patient, such as the presence or absence of the BRCA1 and BRAC2 genes, some have created learning software system that have been trained on these two datasets. In these settings, the systems may also be trained on data relating to prognosis or disease progression, which is information available when conducting retrospective training.

While such multi-dataset machine-learning systems have demonstrated improvements over comparable single modality models, incorporation of different or lager diversity in datasets or modalities, or otherwise deviating from a well-understood process of data analysis conducted by clinicians, is often impractical for a number of reasons. For example, adding additional datasets may include the addition of irrelevant data that is not readily connected to a clear understanding of ground truth. This deviation from retrospective fusion approaches may cause the machine-learning system or model to make incorrect connections and/or hallucinate, both of which are unacceptable in healthcare decision making.

Thus, there is a need for new or different data fusion and machine-learning strategies that can assist, supplement, or supplant traditional clinician analysis or systems that follow traditional clinician analysis.

### SUMMARY

The present disclosure provides systems and methods that overcome the aforementioned drawbacks by incorporating data from a plurality of modalities into a hierarchal structure of machine learning models to construct one or more prognostic signatures of a disease or therapeutic prospects. For example, a hierarchal structure of deep learning networks is provided that provides a multi-scale approach to information analysis across multiple, distinct datasets, such as cellular (genomic), phenotypic (pathology), and structural and functional (MRI) datasets. The systems and methods provided herein can produce a report that includes an integrated marker that is diagnostic of a disease or prognostic of disease outcome, for example, relative to a therapy or treatment.

In one aspect of the present disclosure, a system is provided that includes a computing device. The computing device includes a memory storing instructions and a processor. The processor is configured to access the memory to execute the instructions and, thereby, be caused to receive at least three datasets from differing modalities associated with a disease of a subject and determine a matrix of features for each of the plurality of data modalities. The process is further caused to integrate the matrix of features for each of the data modalities through a sequential, hierarchical structure to create an integrated prognostic signature for the subject and generate a report using the integrated prognostic signature indicating at least one of a diagnosis of the disease or a response of the subject to a treatment. The computing device also includes a display configured to display the report.

In another aspect of the present disclosure, a method is provided that includes receiving, by a system comprising a processor, a plurality of datasets from different modalities associated with a disease of a subject, determining, by the processor, a matrix of features for each of the plurality of data modalities, and integrating, by the processor, the matrix of features through a sequential, hierarchical structure to create an integrated prognostic signature for the subject. The method also includes creating, by the processor, a report using the integrated prognostic signature indicating at least one of a diagnosis of the disease or a response of the subject to a treatment and displaying, by the system, the report.

In one further aspect of the present disclosure, a system for multimodality fusion of medical data sources is provided that include a plurality of modality data sources including a first modality data source including omics data, a second modality data source including histology embeddings, and a third modality data source including radiology data. The system also includes a computing system configured to receive datasets from the plurality of modality data sources, determine a matrix of features for each of the plurality of modality data sources, and integrate the matrix of features of each of the data modalities in a hierarchal learning network to generate a report related to a disease of a subject. The system also includes a display configured to display the report.

These aspects are nonlimiting. Other aspects and features of the systems and methods described herein will be provided below.

### BRIEF DESCRIPTION OF THE DRAWINGS

The foregoing features of embodiments will be more readily understood by reference to the following detailed description, taken with reference to the accompanying drawings, in which:

FIG. 1 is a diagram of a multimodal early fusion system, according to aspects of the present disclosure.

FIG. 2 is a flow chart of the steps of the multi-scale co-attention transformer model, according to aspects of the present disclosure.

FIG. 3 is a schematic block diagram of one, non-limiting example of a hierarchical system in accordance with the present disclosure.

FIG. 4 is a schematic illustration of datasets used in one, non-limiting example test of the system of FIG. 3.

### DETAILED DESCRIPTION

In order to understand disease aggressiveness, assess patient outcomes and associated treatment plans, a routine course of cancer treatments involve acquiring complementary data streams including imaging scans (e.g. CT or MRI images), molecular data (e.g., genome sequencing, gene expression, epigenomics, also known as multi-omics), cellular-scale (e.g., histological images), as well as clinical data (such as age, gender, cognitive scores). Comprehensive assessment of this deluge of data by clinicians alone is infeasible.

The present disclosure recognizes that it would be valuable to develop computational frameworks that integrate complementary cross-scale information across radiology, pathology, genomics, as well as clinical demographics, to enable discovery of new markers that can pave the way for precision medicine. This includes developing integrated prognostic markers that can reliably predict risks associated with clinical outcomes such as survival, recurrence, or disease progression. While initial computational models combining multiple modalities of medical data have demonstrated improvements over comparable single modality models, challenges related to unavailability of multimodality retrospective datasets and careful optimization of fusion strategies are still largely unaddressed. As retrospective studies frequently do not have all data types (radiology, pathology, genomics, demographics) of interest available or correlated in ways that make clinical sense, most existing multimodal approaches have so far only considered two modalities (radiology-pathology, pathology-omics) or involve combing outputs of separate, modality-specific models (i.e. late-stage fusion).

While two modality and late-stage fusion models have demonstrated promise, they do not take full advantage of complementary information and complex multi-scale, cross-correlations across and between diverse data types (i.e. MRI, pathology, genomics). Thus, the present disclosure provides systems and methods for multimodality data analysis that provides greater prognostic power compared to dual modality and/or late-stage fusion approaches. An early fusion approach may be used that can improve the prognostic power when comparing only two modalities that were subject to late-stage fusion.

More particularly, as described herein, late stage fusion combines the outputs of separate models dedicated to a singular data modality at the decision-level. Training such models involves separate processing for each modality being considered, the only aspect that is integrated is the output prediction made for each individual model. On the other hand, early fusion integrates the data within one model, so that correlations and interactions between the different data types can be taken into account when making the overall prediction. Training these models involves inputting all data types or data type representations. The output prediction is based on a numerical representation of how these data types are related to each other and the model task. The present disclosure recognizes that underlying genes influence the pathology and, subsequently, the observed radiology. Thus, the systems and methods provided here use late stage fusion to capture correlations and interactions between these data types in a micro-to-macro approach.

The present disclosure considers a “micro” to “macro” view for building an analysis framework. For example, a “micro” to “macro” view of a tumor reveals that omics data is a close representation of the underlying biological processes, histology images contain rich phenotypic information on how those processes manifest as cellular constructs, and radiology captures large-scale morphology in the context of the affected organ or organ system. Given that measurements at each scale provide unique insight, the present disclosure provides systems and methods for creating and structuring an analysis framework of three or more datasets or modalities. For example, an early fusion data integration model is provided herein to capture a progressive influence from micro to macro datasets (e.g., in this non-limiting example, from genomics to pathology to radiology datasets) and provide report to guide clinical care.

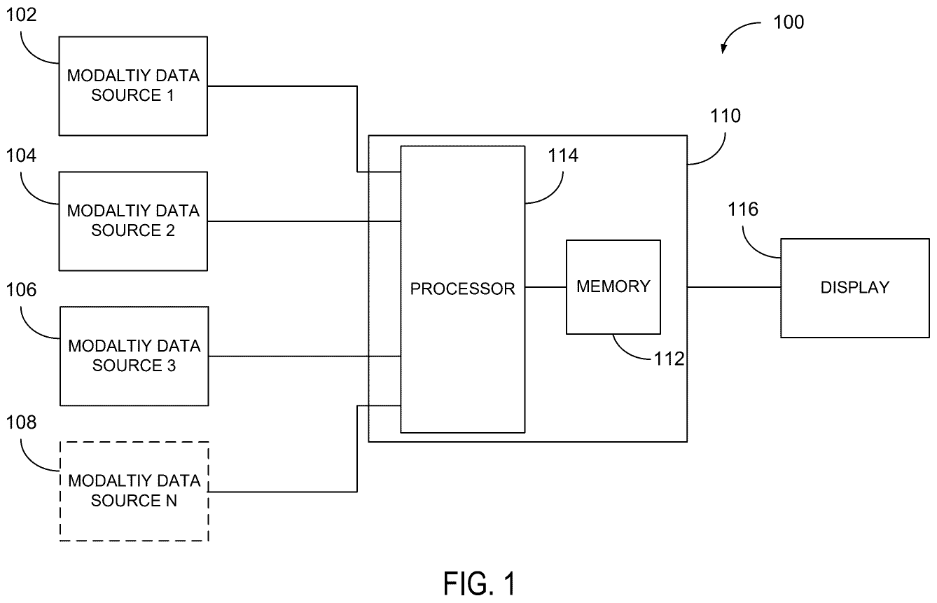

Referring to FIG. 1, one, non-limiting example is provided for an early fusion, multimodality system 100 in accordance with the present disclosure. The system 100 includes a plurality of modality sources data 102-108. For example, the first modality data source 102 may include omics data. The first modality data source 102 may include omics data, such as genomic sequence data including a combination of mutation status, copy number variation (CNV), and mRNA expression data. Omics data may further include epigenomic data. The second modality data source 104 may include histology embeddings. A histology embedding may be a hematoxylin and eosin (H&E)-stained resected tumor whole slide image (WSI). The third data modality source 106 may include radiology data, such as computed tomography (CT) images or magnetic resonance (MR) images. For example, MR images may include pretreatment axial gadolinium-enhanced T1-weighted MR images.

As described, the modality data sources are focused on disease or pathology diagnosis and, in this way, are diagnosis-specific data sources. In a non-limiting example, each of these data modality sources 102-106 may be designed to be specific to a disease or pathology of a subject. For example, the disease may be cancer, such as, but not limited to, glioblastoma, such that the genetic data, the histological data, and the imaging data are all focused on the clinical information used to diagnose or treat glioblastoma.

In a non-limiting example, further modality data source(s) 108 may be included. In one non-limiting example, the further modality data source(s) may include patient data, such as provided in an electronic medical record (EMR). In this way, the further modality data source may include patient or demographic information such as age, sex, and race. The further modality data source 108 may also provide historical information related to the patient, such as prior treatments, resections, and patient outcomes. Such further modality data source 108 may be added to the model as, for example, late fusion data, in the example of certain types of EMR information. Additional data sources could be spatial genomics, immunofluorescence-stained histology slides, various sequences of MRI (T1, T2, T1 with contrast, FLAIR, diffusion weighted, etc.)

In a non-limiting example, the system 100 further includes a computing device 110. The computing device 110 may include a memory 112 for storing instructions thereon and a processor 114 configured to access the memory 112 and execute the instructions. Based on the executed instructions, the processor 114 is configured to communicate to a display 116, for example to communicate a report or other results, as will be described.

The processor 110 is configured to receive information from the modalities data sources 102-018. As will be provided in further detail in the non-limiting example below, the processor 114 can determine a matrix of features for each of the plurality of modality data sources 102-108, and integrate the matrix of features of each of the data modalities in a hierarchal fashion. For example following a “micro” to “macro” view of a condition or disease, the processor 114 may integrate a matrix of the omic data with the histology data first, followed by integrating a resulting matrix with the radiology data.

In a non-limiting example, the processor 114 generates a report or determines an output related to the disease of a subject based on the matrix integrations. The output may be displayed as report that may include a signature, which is communicated to a user via the display 116 operably connected to the computing device 110.

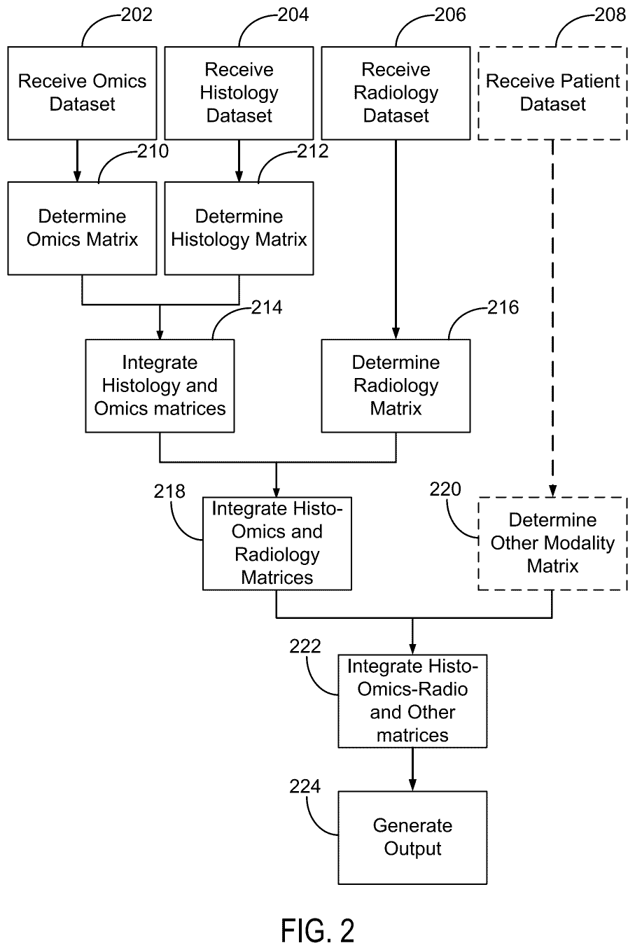

Referring to FIG. 2, a flowchart of non-limiting, example steps performed by the processor 114 of FIG. 1 is provided. At steps 202-208, the processor may receive the first modality dataset (e.g., omics data) 202, the second modality dataset (e.g., histology data) 204, the third modality dataset (e.g., radiology data) 206, and, optionally, patient data 208. It is noted that the plurality of modalities may include any number greater than three. At step 210, the processor determines a matrix of features from the omics data. At step 212, the processor determines a matrix of features from the histology data.

In a non-limiting example, the processor integrates the first modality dataset and the second modality dataset using a multi-scale attention model at step 214. For example, the first co-attention mechanism between the first modality and the second modality can utilize the second modality (e.g., omics data) as a query and the first modality (histology data) as a key and a value of the attention model. In this non-limiting example, omic-directed histology embeddings are produced.

In the non-limiting example following a micro-to-macro hierarchy of omic data to histology data to radiology or imaging data, the processor further determines a matrix of features from the third modality dataset (e.g., radiology data) at step 216. Next, the processor may perform another integration via a second co-attention mechanism using the omic-directed histology embeddings as a query and the third modality (e.g., radiology data) as the key and value of the attention model to produce omic-histology-directed radiology data at step 218. In a non-limiting example, the processor may repeat the determination of a matrix of features for a fourth dataset, a patient dataset, at step 220 and an integration the matrix with the matrix produced in step 218. In this example, at step 222, the processor further separately aggregates the omic data, omic-directed histology data, the omic-histology-directed radiology data, and optionally the omic-histology-radiology-directed patient dataset using respective transformers and global attention pooling.

At step 224, the processor further concatenates the features for each modality, followed by concatenating and input them into three or more fully connected (FC) layers to produce an output. In a non-limiting example, the output may be displayed as a signature including, but not limited to, a hazard score, survival prediction score, or a therapeutic response score.

The above-described systems and methods can be used to implement a “micro” to “macro” hierarchical architecture or framework. In this hierarchical framework views the genomics data as capturing the underlying biological processes, the histology images as containing rich phenotypic information at a cellular level, and the radiology images as reflecting large-scale morphology and structural details. The measurements at each scale provide unique insight. With this in mind, the hierarchical framework provides a multi-modality, early fusion co-attention model to capture the progressive influence from genomics to histology to radiology, using attention mechanisms. The hierarchical or sequentially-informed representations are created by first using an attention mechanism to acquire genomic-directed histology embeddings, which are then used to direct the embeddings of radiology data. These embeddings may then be further processed using mechanism like transformers to capture long-range and complex associations. The multi-scale co-attention transformer (MuSCAT) model is applied to construct integrated prognostic signatures for therapeutic outcome or survival prediction.

The present disclosure recognizes that gradually incorporating complementary information across molecular (genomic) to cellular (histology), to structural and functional (radiology) sources allows the systems and methods provided herein to produce an integrated marker that is highly prognostic of the disease outcome, as compared to a “siloed” approach of interrogating individual data streams.

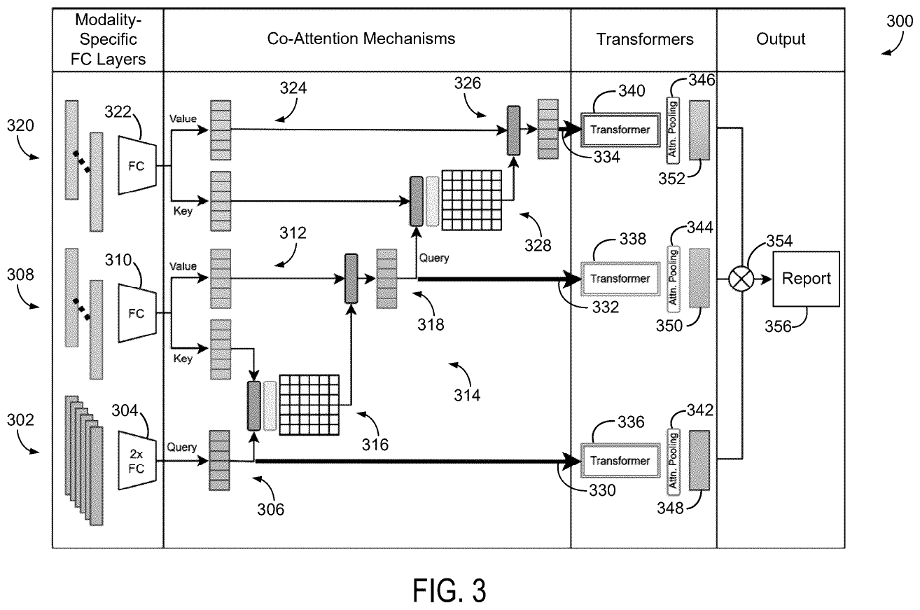

Referring to FIG. 3, one non-limiting example of a system 300 for implementing the above-described hierarchical architecture is provided. In one non-limiting example, the system 300 may realize an early-fusion co-attention transformer framework that incorporates genomics, pathology, and radiology as a multi-scale attention model. In this non-limiting example, the “micro” to “macro” structure begins with genomics data 302. In one non-limiting example, the genomics data 302 may include mutation status, copy number variation, and mRNA expression of cancer-relevant genes, organized into sets by gene family. The genomics data 302 is delivered as the first of the modality-specific fully-connected layer 304. As will be described, to match the sizes of feature embeddings across modalities, the data for each modality is fed into the respective fully connected layer(s) 304, 310, 322. As will be described, queries 306 are performed first on the genomics data 302.

As can be seen in FIG. 3, the genomics data 302 is analyzed first, and then is used to inform the next stage of analysis. That is, the pathology data 308 is provided to a respective fully-connected layer 310. In one, non-limiting example, the pathology data 308 may include patch images that may be extracted from, for example, histology images, and input into the fully-connected network 310. Then the system 300 progressively integrates each data set from the respective modality, starting with a co-attention between genomics data 302 as subject to query 306 and pathology data 308 as subject to key, value embeddings 312, as generally reflected at 314. More particularly, the result may be an attention map 316 to the histology patch embeddings that produces new embeddings containing histology information as pertinent to each gene set 318. In doing so, the dimensionality of the histology images can be reduced from the number of patch images to the number of gene sets.

Next, radiology data 320 is delivered to a respective fully-connected layer 322, to yield value and key outputs 324. The genomic-directed pathology embeddings described above can then be used as the query in a co-attention mechanism between pathology and radiology (key, value) output 324. As one-non-limiting example, the radiology data 320 may include MRI data. Application of the resulting attention map to the MRI slice embeddings can produce new embeddings containing radiology information as pertinent to the histology information of each gene set 326. Again, this mechanism reduces the dimensionality 328 from the number of, for example, MRI slices in the radiology data 320 to the number of gene sets in the genomic data 302.

In addition to the above-described sequential processing, the genomics embeddings 330, genomic-directed pathology embeddings 332, and genomic-pathology-directed radiology embeddings 334 are separately aggregated using respective transformers 336, 338, 340 and global attention pooling 342, 344, 346. The resulting patient-level features 348, 350, 352 for each modality are then concatenated 354 and input into three fully connected layers to produce an output 356. In one, non-limiting example, the report 356 may include a hazard function. In one non-limiting example, a risk score may assign a patient to low, medium, or high risk to survival. A patient with a high risk to their survival may consider more aggressive treatment, for example.

### EXAMPLE

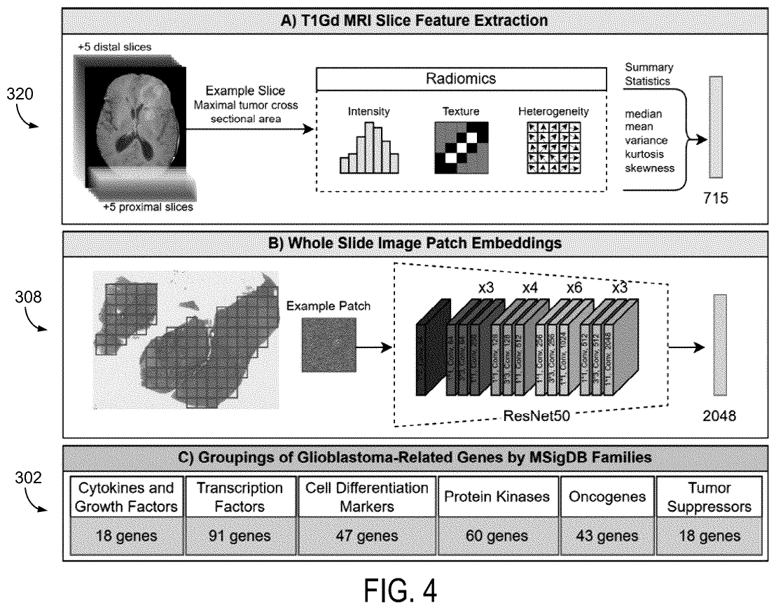

Referring to FIG. 4, in one non-limiting example, the datasets 302, 308, 320 described above were genomic, histology, and MRI data. In particular, genomic, histology, and MRI data was collected from the Cancer Imaging Archive (TCIA), the Cancer Genome Atlas (TCGA), and cBioportal for the publicly available TCGA-Glioblastoma Multiforme (GBM) dataset. In accordance with the 2021 WHO classification of tumors of the central nervous system, cases were categorized as glioblastoma if the patient was IDH-wildtype and presented with one or more of the following: TERT promoter mutation, EGFR amplification, gain chromosome 7 & loss chromosome 10, or a neoplasm histologic grade of 4. Further, patients were only included if structural MRI protocols (Gd-T1w), a radiologist-validated MRI tumor segmentation mask, a diagnostic H&E-stained resected tumor whole slide image (WSI), Affymetrix SNP 6.0 array data, U133a microarray data, and survival information were available. A total of 75 cases had the complete information with all the data streams available and were used in this study.

### Radiomic Feature Analysis on MRI Scans

Pretreatment axial gadolinium-enhanced T1-weighted MR images were employed in the analysis, along with obtaining expert segmentations of the tumor. Preprocessing steps involved isometric re-sampling, registration, skull stripping, and intensity standardization. For each patient, 11 2D MRI slices were selected by first identifying the slice with the largest tumor cross-sectional area. The identified slice as well as the 5 slices immediately proximal and distal were further processed to extract radiomic features capturing intensity, texture, and heterogeneity. Summary statistics including median, mean, variance, kurtosis, and skewness were taken for each feature to produce a 715 feature vector representation for each slice image comprising the MR imaging bag.

Patch-Level Representations from Histology

H&E-stained whole slide images of resected glioblastoma tumors were processed by extracting non-overlapping patch images of stain-normalized tissue. WSIs at 40× magnification were down-sampled to match the remaining cohort at 20× magnification prior to patch extraction at size 256×256 pixels. Embeddings were derived using the clustering-guided contrastive learning (CCL) feature extractor by Wang et al. The CCL feature extractor is a ResNet50 trained using a large database of histopathology images and has demonstrated superior features than those from ResNet models trained using Image-Net. Each patch produced an output 2048 feature vector used for further modeling.

### Genomic Feature Representations

Genomic feature vectors consisted of a combination of mutation status, copy number variation (CNV), and mRNA expression data for genes identified by the Cancer Genome Atlas Network to be involved in signaling pathways of human glioblastoma and those identified by Verhaak et al. to correlate with glioblastoma subtypes (classical, neural, proneural, and mesenchymal). The set of genomic data was organized into 6 feature vectors representing gene families defined by the molecular signatures database to be associated with 1) cytokines and growth factors, 2) transcription factors, 3) cell differentiation markers, 4) protein kinases, 5) oncogenes, and 6) tumor suppressors. Mutation status is categorical and included for 6 cancer-associated genes (EGFR, NF1, PIK3CA, PIK3R1, PTEN, TP53). CNV is the segment mean value derived from the Affymetrix SNP 6.0 array and included for 6 cancer-associated genes (CDK4, CDKN2A, EGFR, MDM4, PDGFRA, PTEN). Lastly, mRNA expression values were represented as expression z-scores relative to diploid samples derived from U133 microarray and include 237 unique genes.

All comparative models (including MuSCAT) were trained using 5-fold cross-validation. To accommodate varying bag sizes of histology patch image feature representations, gradient accumulation was employed with a batch size of 1 and 15 steps for 25 epochs. Due to the singular patient per batch, a discrete survival model consisting of 3 bins was used. The bins were partitioned based on the uncensored survival times in the training set. Zadeh and Schmid's log-likelihood function for deep learning discrete survival modeling was used as the loss function. The concordance index (C-index), defined as the fraction of all pairs of subjects whose predicted survival times are correctly ordered (i.e. concordant with actual survival times), was also computed. C-index=1 indicates that the model has perfect predictive accuracy, and C index=0.5 indicates that the model is no better than random chance.

### Results

Table 1 details the C-indices, computed through 5-fold cross-validation, for the various combinations of the modality feature representation outputs from the respective transformer and global pooling aggregation. Notably, the model, which involved integrating genomic, histologic, and radiology data exhibited superior performance on the validation sets, as compared to the other comparative strategies. The preliminary results suggest that the method may capture prognostic information from each data type, contributing to enhanced survival risk stratification. The observed performance improvement was obtained despite the larger number of trainable parameters corresponding to the network architecture accommodating each data type. Additionally, the results benefited from the use of a histology-specific network for WSI embeddings as well as the adoption of glioblastoma-specific gene sets, as illustrated by the improved performance of the presented genomics and pathology model compared to the genomic and pathology co-attention transformer model developed by Chen et al. (R. J. Chen, M. Y. Lu, W. Weng, et al., “Multimodal coattention transformer for survival prediction in gigapixel whole slide images,” in Proceedings of the IEEE/CVF International Conference on Computer Vision, 2021, pp. 3995-4005; which is incorporated herein by reference.), evaluated on the same dataset.

TABLE 1
C-Index of survival risk predictions for various model
constructions in the training and validation sets.

| | | Training | Validation |
|---|---|---|---|
| Method | Modalities | C-Index | C-Index |
| Presented | Radiology, Histology, | 0.836 (±0.05) | 0.651 (±0.06) |
| Network | Genomics | | |
| | Radiology, Histology | 0.826 (±0.03) | 0.645 (±0.06) |
| | Radiology, Genomics | 0.588 (±0.07) | 0.533 (±0.11) |
| | Histology, Genomics | 0.836 (±0.02) | 0.628 (±0.11) |
| | Radiology | 0.513 (±0.12) | 0.536 (±0.11) |
| | Histology | 0.886 (±0.03) | 0.635 (±0.06) |
| | Genomics | 0.564 (±0.04) | 0.576 (±0.10) |
| Chen et. al | Histology, Genomics | (0.704 ± 0.085) | (0.549 ± 0.09) |

When combining multi-modal genomic, pathology, and radiology data, models may benefit from network architectures that consider influences across modalities. The presented work implemented a new approach for developing integrated risk predictors of disease outcomes, that involved the progressive and systematic combination of genomic, histologic, and radiological data. This work is the first to attempt an early fusion approach across the three modalities in a sequential manner using the co-attention mechanism. Future work will involve evaluating MuSCAT on larger cohort sizes, comparing model performance using alternative deep learning and hand-crafted feature embeddings, and testing our model on independent cohorts.

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

1. A system comprising:
a computing device comprising:
a memory storing instructions;
a processor configured to access the memory to execute the instructions and, thereby, be caused to:
receive at least three datasets from differing modalities associated with a disease of a subject;
determine a matrix of features for each of the plurality of data modalities;
integrate the matrix of features for each of the data modalities through a sequential, hierarchical structure to create an integrated prognostic signature for the subject;
generate a report using the integrated prognostic signature indicating at least one of a diagnosis of the disease or a response of the subject to a treatment; and
a display configured to display the report.

2. The system of claim 1, wherein the at least three datasets from differing modalities comprises a first modality including omics data, a second modality including histology embeddings, and a third modality including radiology data.

3. The system of claim 2, wherein the processor is further caused to integrate the first modality and the second modality using a multi-scale attention model.

4. The system of claim 3, wherein the processor is further caused to perform a first co-attention mechanism using the omics data as a query and the histology data as a key and a value of the attention model to produce omic-directed histology embeddings.

5. The system of claim 4, wherein the processor is further caused to perform a second co-attention mechanism using the omic-directed histology embeddings as a query and the radiology data as a key and a value of the attention model to produce omic-histology-directed radiology data.

6. The system of claim 5, wherein the processor is further caused to separately aggregate, using a transformer for each, the omic data, omic-directed histology embeddings, and the omic-histology-directed data via global attention pooling to produce one or more features for each of the plurality of data modalities.

7. The system of claim 6, wherein the processor is further caused to concatenate the one or more features and into a plurality of fully connected layers to produce the report.

8. The system of claim 2, wherein the histology embeddings include whole slide image patch embeddings of a disease sample.

9. The system of claim 2, wherein the omics data includes at least one of genome sequencing data, gene-expression data, and epigenomics data.

10. The system of claim 2, wherein the radiology data includes imaging slices.

11. The system of claim 10, wherein the imaging scans include at least one of computed tomography (CT) or magnetic resonance (MR) images.

12. The system of claim 2, wherein the at least three datasets from differing modalities further comprises a fourth modality including patient data.

13. The system of claim 1, wherein the signature includes at least one of a hazard score, survival prediction score, or a therapeutic response score.

14. A method comprising:
receiving, by a system comprising a processor, a plurality of datasets from different modalities associated with a disease of a subject;
determining, by the processor, a matrix of features for each of the plurality of data modalities;
integrating, by the processor, the matrix of features through a sequential, hierarchical structure to create an integrated prognostic signature for the subject;
creating, by the processor, a report using the integrated prognostic signature indicating at least one of a diagnosis of the disease or a response of the subject to a treatment; and
displaying, by the system, the report.

15. The method of claim 14, wherein the plurality of datasets comprises a first modality dataset including omics data, a second modality dataset including histology embeddings, and a third modality dataset including radiology data.

16. The method of claim 15, further comprising integrating, by the processor, the first dataset modality and the second modality dataset using a multi-scale attention model.

17. The method of claim 16, further comprising performing, by the processor, a first co-attention mechanism using the omics data as a query and the histology data as a key and a value of the attention model to produce omic-directed histology embeddings.

18. The method of claim 17, further comprising performing, by the processor, a second co-attention mechanism using the omic-directed histology embeddings as a query and the radiology data as a key and a value of the attention model to produce omic-histology-directed radiology data.

19. The method of claim 18, further comprising, by the processor, separately aggregating, using a transformer for each, the omic data, omic-directed histology embeddings, and the omic-histology-directed data via global attention pooling to produce one or more features for each of the plurality of data modalities.

20. The method of claim 19, further comprising, by the processor, concatenating the one or more features and into a plurality of fully connected layers to produce the integrated prognostic signature.

21. The method of claim 15, wherein the plurality of datasets from differing modalities further comprises a fourth modality including patient data.

22. A system for multimodality fusion of medical data sources, comprising:
a plurality of modality data sources including a first modality data source including omics data, a second modality data source including histology embeddings, and a third modality data source including radiology data;
a computing system configured to:
receive datasets from the plurality of modality data sources;
determine a matrix of features for each of the plurality of modality data sources; and
integrate the matrix of features of each of the data modalities in a hierarchal learning network to generate a report related to a disease of a subject; and
a display configured to display the report.

23. The system of claim 22, wherein:
the omics data comprises at least one of genome sequencing data, gene-expression data, and epigenomics data;
the histology data comprises hematoxylin and eosin-stained resected tumor whole slide images; and
the radiology data comprises at least one of computed tomography images and magnetic resonance images.

24. The system of claim 22, wherein the report that includes an integrated marker that is diagnostic of a disease or prognostic of disease outcome.

25. The system of claim 22, wherein the computing system is further configured to integrate the matrix of features of each dataset received from plurality of modality data in a hierarchal fashion following a micro-to-macro view of a condition or disease.

26. The system of claim 22, wherein the computing system is further configured to determine a matrix of features for a fourth modality data source comprising patient data, and integrate the matrix of features of the fourth modality data source with the matrix integrations of the first, second, and third modality data sources.
